(19) United States
(12) Patent Application Publication
(10) Pub. No.: US 2011/0127177 A1
(43) Pub. Date: Jun. 2, 2011

(54) CONTAINER FOR RECEIVING LAB SAMPLES, AND USE OF A DEVICE FOR TAKING SAMPLES

(75) Inventors: Bernhard Hostettler, Gockhausen (CH); Brigitta Kipfer-Schafer, Rheinfelden (CH); Christoph Stamm, Stein am Rhein (CH)

(73) Assignee: PRIONICS AG, Schlieren (CH)

(21) Appl. No.: 13/056,193

(22) PCT Filed: Jul. 28, 2009
(86) PCT No.: PCT/EP09/05452

§ 371 Date: Jan. 27, 2011

(30) Foreign Application Priority Data
Aug. 1, 2008 (DE) 10 2008 035 851.7

(51) Int. Cl. B65D 81/26; A61B 5/00; B29C 45/14

(52) U.S. Cl. 206/204; 600/582; 264/254

(57) ABSTRACT

Container for receiving lab samples, whereby the samples are held in the container at a distance from the internal wall and in a defined height range, and whereby a drying device is provided inside the container at least in said height range and surrounds the sample on the side, at least partially, and includes a drying agent, and is constructed such that gas exchange between sample and drying agent is feasible.

The invention relates to a container for receiving lab samples, which can be used, in particular, in forensic securing of evidence, but is also well-suited for receiving other lab samples, e.g., in the fields of medicine or molecular biology or the like.

Usually sampling is made using a set consisting of a test tube and a rod that is held by a stopper or other closure that fits on the test tube, whereby the rod has a cotton swab or the like attached to its free end for taking samples, e.g. a buccal smear. The test tube and the rod are matched to each other such that the closure can be placed on the test tube, whereby the rod then extends into the interior of the test tube and its free end-region, intended for taking samples, e.g. the cotton swab, is held in a defined height range of the test tube and usually without contact to the internal wall of the test tube.

Usually a sample is not processed immediately after it is taken. Rather, for a number of reasons, some time elapses before the sample is processed. In particular in the field of securing of evidence, but, as a matter of fact, in genetic or microbiological tests as well, it is particularly important for the sample to not undergo any changes between sample-taking and processing. Changes in the sample material must be expected to occur in particular if the sample is moist and not chilled.

With regard to this problem, it is known to provide drying agents in containers for receiving lab samples, which drying agents are intended to gradually remove water from the sample that was taken. In this regard, reference is made to WO 2007/029025 which shows a sample-taking container that has drying agent arranged in its bottom region. It is also mentioned therein that the internal walls of the container may be coated with drying agent.

WO 00/51496 shows a system, in which a sample-taking test tube comprises two compartments in longitudinal direction, namely a lower compartment filled with drying agent and an upper compartment, in which the sample is arranged. A similar device is shown by GB 2358061.

It has turned out that the desired drying rate cannot be attained using the known systems. Customarily, it is desirable in forensic applications to have the cotton swab used to take a sample dry quickly enough such as to prevent the growth of microorganisms.

It is the object of the invention to create a container for receiving lab samples based on the prior art, in which container more rapid and more effective drying is feasible as compared to known systems.

Said object is met by a container according to claim 1.

In the container according to the invention, a drying unit that surrounds the sample, at least partly, on the side is provided at least in the height range, in which the sample is arranged in the container once it has been taken.

In the context of the present application, the term, sample, shall comprise not only the actual sample material, but also the sample-taking device that contains the sample material, e.g. the cotton swab at the end of the holding rod mentioned above.

The drying unit contains drying agent and is provided such that the drying agent contained therein can undergo gas exchange with the sample, e.g. with a cotton swab soaked in sample fluid, and then dry the sample. The drying agent can be, e.g., hygroscopic substances, e.g. zeolite, silica gel or molecular sieve, to name but a few examples.

It has been evident that, unlike with known systems, the solution according to the invention is associated with effective and rapid drying of the sample. Verified by measurements, this is related to the relatively close arrangement of sample and drying agent as provided for by the invention. In contrast, there is a relatively large distance between drying agent and sample in the known systems.

Both the known system and the system according to the invention are closed systems, i.e. once the sample has been taken, the container comprising the closure holding the rod-shaped sample-taking device is closed in an air-tight manner. The air space in a container of this type that is sealed in an air-tight manner can be presumed to be stagnant, i.e. gas exchange proceeds by means of diffusion only and therefore only to a small extent and slowly over larger distances.

The same is not the case with the container according to the invention. Here, targeted and specific arrangement of the drying agent in a region of the container that is right next to the sample is feasible which leads to successful drying.

Advantageous further embodiments of the invention are specified in the dependent claims.

A first advantageous embodiment provides the drying unit to be a molded part that contains the hygroscopic substance. It is conceivable, e.g., that the hygroscopic substance is embedded in or applied to a plastic material from which, e.g., a film material can then be produced that represents the drying unit after it is inserted or placed in the sample container.

The prior art knows plastic materials of this type that can be produced, e.g., by injection molding, and can include up to 70% hygroscopic substance (e.g. molecular sieve). Obviously, it is also conceivable to produce drying units of other suitable shapes by injection molding from said plastic materials, and use them thereafter.

It is self-evident that the scope of the present invention also includes production of the drying unit and container in a 2-step injection molding procedure by means of a method according to claim 9. It would be conceivable, e.g., to use a special tool to first produce a tube-shaped drying unit by injection molding and then to provide the container around it. In this case, the drying unit would not be produced as a separate insert.

Aside from the above-mentioned injection molding procedure, other procedures, such as, e.g., extruding, punching or rolling, are feasible as well, which is self-evident.

Obviously, it is also feasible in further embodiments for the drying unit to be provided by an insert that is inserted into the container and comprises a wall region and a bottom region and defines in the container an external circumferential compartment for receiving the drying agent and an internal compartment for the sample. The wall region is provided with perforations that facilitate gas exchange between drying agent and sample, while they particularly preferably prevent the usually particulate drying agent from passing through them.

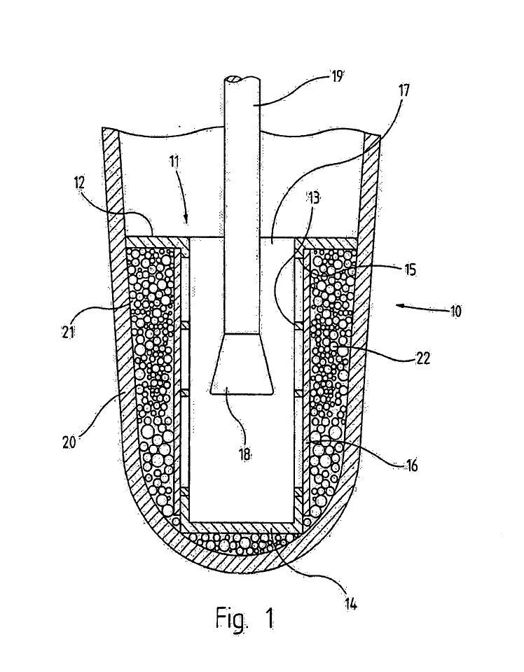

The containers according to the invention usually are test tubes. A conceivable insert therefore might take the shape of a cylindrical sleeve with a smaller diameter as compared to the test tube that is furnished at its upper end (with respect to the entry opening of the test tube) with a circumferential collar with an external diameter that corresponds to the internal diameter of the test tube. Accordingly, the collar would prevent drying agent to exit upwards into the test tube. In this context reference is made to FIG. 1.

As mentioned above, the wall region of the insert is provided with perforations which have special properties. This can, under certain circumstances, be a problem during the production thereof, if the insert is a plastic part. Therefore, a further embodiment of the invention provides the wall regions of the insert with arbitrary perforations, whereby, in addition, a device covering the inside or the outside of the wall region is provided that generates the specific permeability properties. The device can, e.g., be a type of fine-pored coating that is applied from outside to the wall region of the insert or, alternatively, on the inside thereof. In this context, the pore size is preferably selected such that it allows for gas ex-change between drying agent and sample, but prevents drying agent particles from passing through to the sample space.

The production of the container according to the invention of the embodiment just discussed proceeds as follows:

Firstly, the particulate drying agent is filled into the test tube. Then, the insert, which is possibly provided with the above-mentioned coating, is inserted into the test tube, but not yet slid into its final position. In a next step, the test tube is turned upside-down upon which the drying agent distributes throughout the external compartment that is defined by the insert. Then the insert is slid into its final position.

This type of production is relatively laborious.

Accordingly, further embodiments of the invention provide drying units that can be provided with drying agent outside of the test tube and are then inserted into the test tube as a unit.

In this context, a particularly preferred embodiment provides the drying agent to be formed into a solid, e.g. sleeve-shaped, drying unit that can be inserted into the container without any need for further inserts, etc. It is conceivable to compact, sinter or extrude the drying agent.

Obviously, as has already been mentioned above, the drying agent can be pro-vided to be embedded in a plastic material from which, e.g., a film material can be produced that can be inserted or placed in the container as drying unit. Self-evidently, it is also conceivable to produce drying units having other suitable shapes from said plastic materials by injection molding, and then insert them.

Another particularly preferred embodiment provides the drying unit to be a de-vice that includes a bottom region, an external circumferential wall region, and an internal circumferential wall region. Analogous to the insert discussed above, this defines between the internal and the external wall region a compartment for the drying agent, and by means of the internal wall region, a compartment for receiving the sample. The external compartment is then filled with the drying agent and closed on the top. The device can then be inserted into the container. It is evident that the internal wall region includes the required perforations for the desired gas exchange between sample material and drying agent, as before. Obviously, the coating or a corresponding lining of the internal wall mentioned above can be provided in this case as well. The container according to the invention having the device just mentioned is easier to produce as compared to the embodiment comprising the insert, whereby this solution also ensures that the surface area and thus the drying activity of the drying agent are maximal.

The invention relates not only to a container for receiving lab samples, but also to the use of a device for taking samples.

In particular in the field of forensics, samples may need to be taken at sites that are difficult to access (e.g. under the fingernail) or samples may have dried up and cannot be taken with the cotton swab that is used customarily.

The invention provides for this case to use a sample-taking device that includes a holding rod that extends from a stopper and whose cross-section tapers towards its free end such that an edge is produced, and which is provided in said end-region with a flocking that takes up the sample material. The invention can also provide for the length of the rod to be shorter than in the devices that are customarily used for taking samples. Alternatively or in addition, the invention can pro-vide for the rod as well as the flocking to be relatively rigid such that dried up samples can also be taken and/or broken up. The flocking can, e.g., be a hardened flocking comprising cotton.

Devices of this type are basically known from the field of cosmetic flocking. However, they have not been applied yet in the field of forensics.

Said devices can be used, in particular, in sets comprising the containers according to the invention.

The invention shall be illustrated in more detail in the following by means of several drawings. In the figures:

FIG. 1 shows a sectional view of a test tube, in which the drying unit is provided by means of an insert.

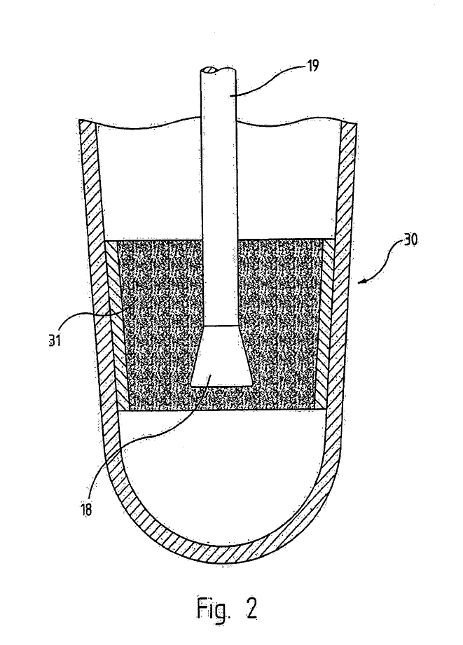

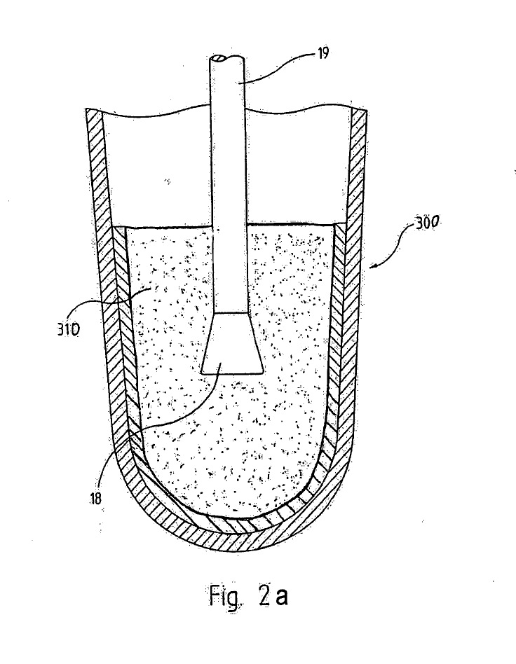

FIG. 2 shows a sectional view of another exemplary embodiment of the test tube according to the invention, in which the drying unit is a plastic film/drying agent film arranged on the inside.

FIG. 2a shows a sectional view of another exemplary embodiment of the test tube according to the invention, in which the drying unit is a plastic part in the form of a test tube that is provided on the inside.

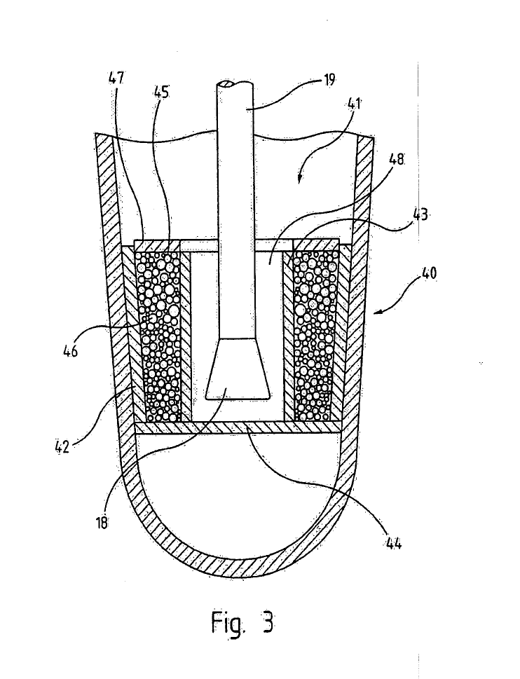

FIG. 3 shows a sectional view of an exemplary embodiment of the container according to the invention, in which the drying unit is a de-vice that is produced separately and can then be inserted into the container.

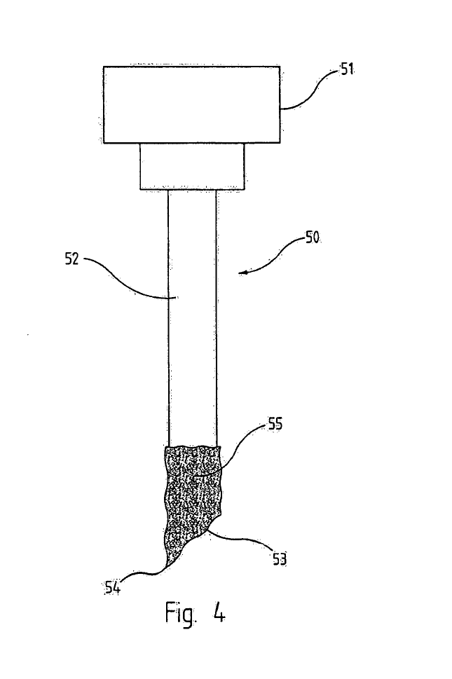

FIG. 4 shows a lateral view of the lower end of a sample-taking device for difficult samples.

FIG. 1 shows the lower part of a container 10 in a sectional view. In the case shown, the container is a test tube which is customary. A drying unit 11 in the form of an insert is arranged in the container and includes an upper circumferential collar 12 and, extending downwards from there, a wall region 13 as well as a bottom region 14. Perforations 15 are provided in said wall region 13. A coating 16 is provided on the inside or outside of said wall region 13. This coating can, e.g., be an elastic film, a textile material, a fleece or the like. The purpose of coating 16 is to form a defined boundary layer that permits passage of gas molecules (e.g. water molecules), but prevents larger particles, e.g. abraded material or dust of the drying agent, from passing through.

The wall region 13 defines an internal compartment 17, in which the lower, sample-receiving end 18 of a sample-taking device 19 is arranged in the case shown here.

Moreover, wall region 13 and/or the coating 16 arranged thereon and a wall region 20 of the container 10 jointly define an external compartment 21, in which drying agent 22 is arranged. It is quite evident from the figure that the drying agent 22 is situated in immediate proximity to the end 18 of the sample-taking device 19 that is intended for taking the sample in the exemplary embodiment shown. This ensures optimal drying. As mentioned above, this end 18 shall also be encompassed by the term, sample. Accordingly, at least in the exemplary embodiments shown, the task of the drying agent is to dry the sample-containing end, e.g. a cotton swab, and thus obviously simultaneously dry the lab sample contained therein or thereon.

As mentioned already above, the production of the exemplary embodiment shown in FIG. 1 necessitates a number of steps.

A simplified production is enabled by the exemplary embodiments shown in FIGS. 2, 2a and/or 3.

FIG. 2 shows the lower region of a container 30, as before. A film 31 containing drying agent is placed in the container and partially lines the internal wall.

In analogy to the exemplary embodiment shown in FIG. 2, it would also be conceivable to compact, extrude or sinter the drying agent into a drying unit having the desired shape. A drying unit produced as described could be produced separately, like the film 31 shown in FIG. 2, and then inserted into a container. No drawing is shown here in this context.

FIG. 2a shows a sample container 300, in which a drying unit 310 is provided in the form of a plastic test tube. It is self-evident that the plastic test tube contains the hygroscopic substance in this embodiment as well. This embodiment is advantageous in that it is particularly easy to produce by means of a, e.g. two-step, injection molding process including a specialized tool. In a first step, e.g., the internal plastic test tube containing the hygroscopic substance is produced by injection molding. Subsequently, the external container is produced around the internal test tube by injection molding.

FIG. 3 shows the lower region of a test tube 40, as before. A drying unit device 41 is inserted into the test tube 40 and comprises an external circumferential wall region 42, an internal circumferential wall region 43 as well as a bottom region 44. The external wall region and the internal wall region 43 define an external compartment 45, in which drying agent 46 is arranged. Said compartment 45 can be closed in upward direction after filling it using a ring-shaped closure 47. Moreover, the wall region 43 defines an internal compartment 48, in which the sample is arranged, which is analogous to the exemplary embodiments described thus far. What is not shown, but is self-evident to also be provided in the present exemplary embodiment is that perforations are present in said internal wall region 43 and permit gas exchange between sample and drying agent 46. Obviously, the coating of the internal or external surface as discussed above may be used in this exemplary embodiment as well.

FIG. 4 schematically shows the above-mentioned device 50 for taking difficult samples. In the exemplary embodiment shown here, the device includes a rod 52 that is held by a stopper 51 and is cut off obliquely on its free end 53. From this results an edge 54. Moreover, the rod 52 is provided with flocking 55 on its free end 53 for receiving the sample. The device shown is particularly well-suited for taking samples from under fingernails or other sites that are difficult to access. Moreover, said device allows, e.g., dried up samples to be taken.

The stopper can be used to insert said device in the above-mentioned containers for storage and/or transport, whereby the containers preferably include a drying unit according to the invention.

1. A container for receiving lab samples, whereby the samples are held in the container at a distance from an internal wall and in a defined height range, wherein a drying device, which includes a drying agent, is provided inside the container at least in said height range and surrounding the sample at least partially on the side, such that a gas exchange between sample and drying agent is possible.

2. The container according to claim 1, wherein the drying device is a plastic molded part that contains the drying agent.

3. The container according to claim 1, wherein the drying device is an insert comprising a wall region that forms an external compartment and an internal compartment in the container, whereby the external compartment is intended to be filled with drying agent and perforations enabling gas exchange between sample and drying agent are provided in the wall region.

4. The container according to claim 3, wherein a coating is provided, that covers the wall region on the inside and/or outside and defines a boundary layer that allows water molecules in the gas phase to pass through it, but prevents larger particles from passing through it.

5. The container according to claim 1, wherein the drying device and the drying agent contained therein jointly are provided in the form of a single component that can be inserted into the container.

6. The container according to claim 5, wherein the drying device is a drying agent that is made into a desired shape.

7. The container according to claim 5, wherein the drying device includes an external wall region an internal wall region, and a bottom region, whereby the two wall regions and form an external compartment that can be closed by means of a ring-shaped closure and can be filled with drying agent.

8. A device for sample-taking, consisting of a rod that is held by a container closure and whose free end is cut off obliquely such that an edge is formed and which is provided with a flocking at its free end.

9. A method for producing the container according to claim 2, comprising producing the drying device and the container in a two-step injection molding procedure.

10. The container according to claim 6, wherein the drying agent is made into the desired shape by means of compacting, sintering or extruding.
